(19) United States
(12) Patent Application Publication
(10) Pub. No.: US 2012/0089197 A1
(43) Pub. Date: Apr. 12, 2012

(54) ARTHRODESIS IMPLANT APPARATUS AND METHOD

(76) Inventor: Gregory S. Anderson, Sandy, UT (US)

(21) Appl. No.: 12/901,552

(22) Filed: Oct. 10, 2010

(51) Int. Cl. A61B 17/86

(52) U.S. Cl. 606/310

(57) ABSTRACT

An arthrodesis anchor is formed as a monolithic piece for ease of handling and use, having two ends, one of which has prongs supporting barbs extending radially therefrom. The other end may have threads or barbs. A neck between may circular in cross section and be sized from about 0.045 inches to about 0.08 inches and may be conveniently sized at 0.062 inches for bending to accommodate final alignment of joints to be bonded. Optionally, a shank next to the neck may receive a tool for threading the anchor into a joint. The proximal and intermediate phalangial joints may be trimmed and pilot drilled, after which a first end may be inserted by a tool, typically into the proximal joint by threading or linear insertion of barbed prongs. The second end having barbed prongs may then be inserted into a pilot drilled into the base of the intermediate joint.

### BACKGROUND

1. The Field of the Invention

This invention relates to orthopedic surgery and more particularly to methods and apparatus for fixing bone elements in suitable positions for permanent healing.

2. The Background Art

Orthopedic surgeries originated millennia ago. Anthropological excavations have demonstrated orthopedics practices evidenced in healed, sometimes structurally reinforced, bone healing. Modern orthopedic surgery has greatly advanced the art of improving deformities through surgery. Moreover, the importance of maintaining an active life has motivated demonstrably improved methods for promoting the ability to heal.

For example, patients with a variety of recovery needs were customarily committed to bed rest in hospitals in decades past. Modern medicine realizes the emotional and physical toll that such inactivity takes on a patient. Modern surgical techniques acknowledge the importance of maintaining a physically active body as a mechanism to aid in healing processes. Accordingly, it is desirable to have patients continue in their daily movement and activities as soon as possible. To this end, less invasive surgical techniques have been developed.

Likewise, structural implants such as plates, screws, staples, rods, pins and the like have been augmented by new joint systems, and other orthopedic implants to replace or enhance natural orthopedic structures in the body.

Nevertheless, surgical techniques need to be simplified in many instances. Likewise, the speed at which surgical procedures can proceed is limited by both the physical circumstances of the injury or malady being corrected, as well as the equipment used, and any other supporting equipment or devices required by the procedure.

Accordingly, it would be an advance in the art to provide implants, such as orienting pins, screws, attachments, and the like in a way that provides faster installation, more secure holding, post-installation adjustment, and the like. Such features would provide to a surgeon the additional benefit of being able to confirm securement, adjust angles of relative positioning between adjacent bone elements, and do so late in the procedure, as a feedback-controlled final adjustment.

For example, it would be an advance in the art if a surgeon were able to adjust the angle of two bones being conjoined for a hammertoe surgery Likewise, it would be an advance in the art to provide robust, strong, and yet bendable or otherwise adjustable portions of a bone anchor in order that a doctor could survey his handiwork near the close of an operation, and readjust angles, proximities, and the like before closing up the incisions.

It would be a further advance in the art if the attachment mechanisms for such an inter-element anchor were sufficiently strong and engaged that the bone elements being anchored could themselves serve as the handles in order to support the final orientation thereof after the anchor has been placed. It would also be an advance if the anchor could support three dimensional stability following surgery in order to hasten healing, permit early use promoting circulation and healing, and otherwise provide securement with less threat of separation, twisting, disorientation, and the like during the important early days of the healing process.

It would be a further advance of the art to provide three dimensional stabilization between two elements being joined in an orthopedic, and particularly a hammertoe remediation, by providing reliable anchoring in the longitudinal direction, as well as orientation in the lateral and transverse directions orthogonal thereto in order that healing begin early and be promoted by stability of the joint in all three dimensions.

### BRIEF SUMMARY OF THE INVENTION

In view of the foregoing, in accordance with the invention as embodied and broadly described herein, a method and apparatus are disclosed in one embodiment of the present invention as including a method for controlling application of an anchor may be based on facts corresponding to the entity controlling the application.

In one embodiment of an apparatus and method in accordance with the invention an orthopedic anchor, particularly one adapted to arthrodesis, may include two ends of a single, solid member. A first end may be threaded with flutes adapted to be threaded into a pilot hole formed to extend into the medullar portion of a bone segment. Typical bone segments may include the proximal and intermediate phalangial joints of a patient, such as a patient having a hammertoe deformity.

The flutes of the threads may be sized in pitch and depth to promote gripping of the flutes against not only the structural portion of the marrow or medullar portion of the bone segment into which penetrating, but also to engage the cortical portion of the bone in order to provide a secure longitudinal attachment. Accordingly, the length and diameter may be sized to engage the cortical region, and the placement may be selected in order to optimize this engagement.

The opposite end of the anchor may be formed as an array of barbed flutes providing a substantially rectangular cross section. As a practical matter, the barbs may actually be considered circular, but typically having flats formed in diametrically opposed sides. Thus, the fluted, arcuate portions of the barbs may extend laterally in one dimension, but be absent at ninety degrees therefrom.

Likewise, a slot may be formed in the barbed end of the anchor to extend between the two flat aspects of the barbed end. In this way, a tool may be fitted against the flats, having a web connecting between the flats, and extending through the slot formed between the barbs. Various other webs may be formed in tool or barbs in order to stabilize the two prongs or legs of the barbed end with respect to one another, minimize the unsupported length thereof, and the like.

Between the threaded or screw end of the anchor and the barbed end thereof, a hexagonal or other tool-shaped portion or shank may extend. This portion may permit or admit access by a tool for turning the anchor once in place Likewise, engagement by a tool for driving may also be supported by a shaped, typically a hexagonal, cross section thereof.

In one apparatus and method in accordance with the invention, the tool-receiver portions of the shaped (e.g., hexagonal) portion proximate the screw end on the anchor and the slotted driver-receiver portion between the flutes of the barbed end of the anchor may include a neck portion having the minimum diameter available in the anchor. Accordingly, this neck portion, having the minimal diameter, therefore has the smallest section modulus along the length of the anchor. This portion, when formed to be of circular cross section, has the ability to be angled in any direction of the axial center or longitudinal axis.

Thus, a surgeon may put a bend in the neck of the anchor prior to the final installation, or at any time. In one embodiment of a method in accordance with the invention, a surgeon may maintain the anchor completely straight throughout the surgery, and then provide any angular adjustment in any desired direction by bending the anchor in that direction.

Accordingly, a surgeon may maintain surface alignment and contact between the resected contact faces of the bone elements being joined Likewise, those faces may be canted slightly with respect to one another in order to maintain the desired orientation, thus relying on the bones to fill in any misalignments of the faces, in order to support longitudinal alignment of the bone elements in this surgical procedure.

In certain embodiments of an apparatus and method in accordance with the invention, an anchor may be pre-angled between the barbed end and the threaded end. In such an embodiment, a tool may be provided that is exactly matched to any “misalignment” of the angle of the barb with respect to the longitudinal axis of the screw. In such an embodiment, for example, the tool may have matching angles provided.

The front end and engagement portion of the tool first angles away from the longitudinal axis of the tool, in order to obtain a distance of angle required to support the angle of the barb. Then, the tool angles again directly down the length of the barb, thus providing axial alignment between the handle of the tool and the longitudinal axis of the screw end.

In this way, the tool provides a “dogleg” effect in which the screw is axially aligned along a longitudinal axis, and the handle is collinear therewith. Meanwhile, between the handle of the tool and the threaded end of the anchor, a dogleg angles out away from the longitudinal axis, and then back in towards the longitudinal axis, aligned exactly with the longitudinal axis of the barbed portion by which the tool engages the anchor. Meanwhile, the handle end of the tool aligns at all times with the screw.

### BRIEF DESCRIPTION OF THE DRAWINGS

The foregoing features of the present invention will become more fully apparent from the following description and appended claims, taken in conjunction with the accompanying drawings. Understanding that these drawings depict only typical embodiments of the invention and are, therefore, not to be considered limiting of its scope, the invention will be described with additional specificity and detail through use of the accompanying drawings in which:

FIG. 1 is a perspective view of one embodiment of an apparatus in accordance with the invention including both an anchor and a tool for manipulating the anchor during initial stages of the installation process;

FIG. 2 is a perspective view of the anchor and tool of FIG. 1, viewed from the opposite end thereof;

FIG. 3 is a top plan view of the anchor of FIG. 1;

FIG. 4 is a bottom plan view thereof;

FIG. 5 is a left side elevation view thereof;

FIG. 6 is a right side elevation view thereof;

FIG. 7 is an end elevation view from the screw point end;

FIG. 8 is an end elevation view thereof showing the pronged or barbed end that is engaged by the tool;

FIG. 9 is a perspective view of one embodiment of the tool of FIG. 1, having the center portion shortened as indicated in order to provide detail of the handle and anchor ends thereof;

FIG. 10 is a top plan view of the tool of FIG. 9;

FIG. 11 is a bottom plan view thereof;

FIG. 12 is a left side elevation view thereof;

FIG. 13 is a right side elevation view thereof;

FIG. 14 is an end elevation view of the screw engagement end of the tool of FIGS. 1 and 9-13;

FIG. 15 is an end elevation view of the tool of FIGS. 1 and 9-14, from the handle-end thereof;

FIG. 16 is a perspective view of the anchor of FIGS. 1-6 showing the process of engagement thereof with a bone joint;

FIG. 17 is a perspective view of the anchor of FIG. 16, as the barbed end thereof is inserted into the bone joint that is to be fused with the first bone joint;

FIG. 18A is a perspective view of an alternative embodiment of the tool of FIGS. 1 and 9, designed to accommodate threading of the screw portion of the anchor into a bone joint when the anchor has been formed with a pre-defined bend angle between the barbed portion and the threaded portion of the anchor;

FIG. 18B is a side elevation view thereof with an anchor in place ready for insertion;

FIG. 18C is a side elevation view of an alternative embodiment of the tool of FIGS. 18A-18B;

FIG. 19 is a perspective view of an alternative embodiment of an anchor in accordance with the invention, not relying on a hexagonal shank;

FIG. 20 is a perspective view from the opposite end thereof;

FIG. 21 is a top plan view thereof;

FIG. 22 is a bottom plan view thereof;

FIG. 23 is a left side elevation view thereof;

FIG. 24 is a right side elevation view thereof;

FIG. 25 is an end elevation view from the screw end thereof;

FIG. 26 is an end elevation view from the barbed end thereof;

FIG. 27 is a perspective view of an alternative embodiment of an anchor in accordance with the invention relying on two barbed ends rather than a screw;

FIG. 28 is a perspective view thereof from the opposite end, and rotated at right angles about a longitudinal axis;

FIG. 29 is a top plan view of the embodiment of FIG. 27;

FIG. 30 is a bottom plan view thereof;

FIG. 31 is a left side elevation view thereof;

FIG. 32 is a right side elevation view thereof;

FIG. 33 is an end elevation view thereof from the back end having fewer barbs;

FIG. 34 is an end elevation view from the opposite end thereof;

FIG. 35 is a perspective view of an alternative embodiment of an anchor in accordance with the invention relying on two barbed ends, and having a pre-formed bend angle between the longitudinal axes thereof;

FIG. 36 is a perspective view thereof from the opposite end, and rotated at right angles about a longitudinal axis;

FIG. 37 is a top plan view of the embodiment of FIG. 35;

FIG. 38 is a bottom plan view thereof;

FIG. 39 is a left side elevation view thereof;

FIG. 40 is a right side elevation view thereof;

FIG. 41 is an end elevation view thereof from the back end having fewer barbs; and

FIG. 42 is an end elevation view from the opposite end thereof.

### DETAILED DESCRIPTION OF THE PREFERRED EMBODIMENTS

It will be readily understood that the components of the present invention, as generally described and illustrated in the drawings herein, could be arranged and designed in a wide variety of different configurations. Thus, the following more detailed description of the embodiments of the system and method of the present invention, as represented in the drawings, is not intended to limit the scope of the invention, as claimed, but is merely representative of various embodiments of the invention. The illustrated embodiments of the invention will be best understood by reference to the drawings, wherein like parts are designated by like numerals throughout.

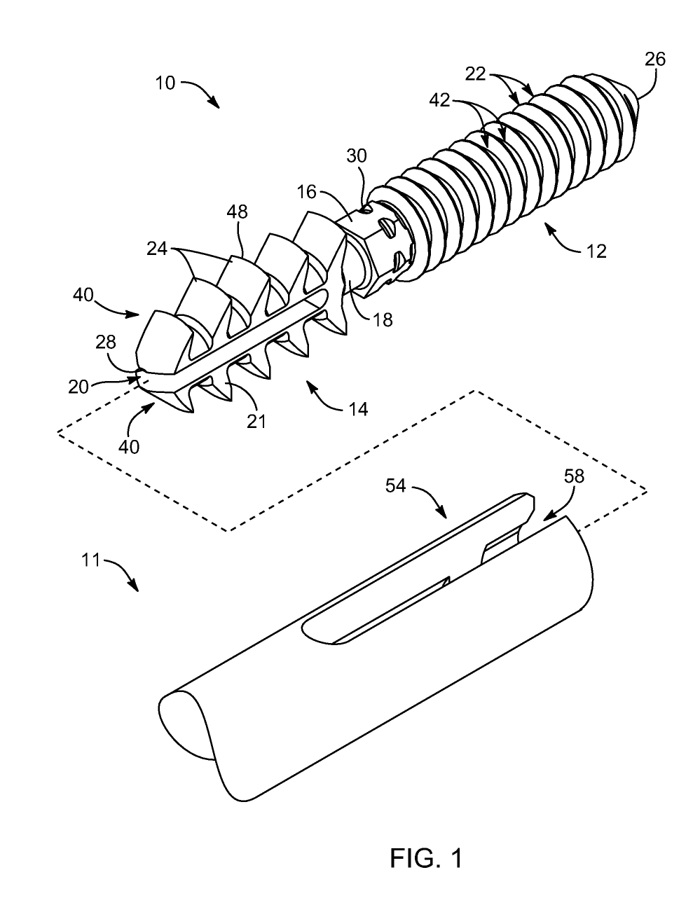

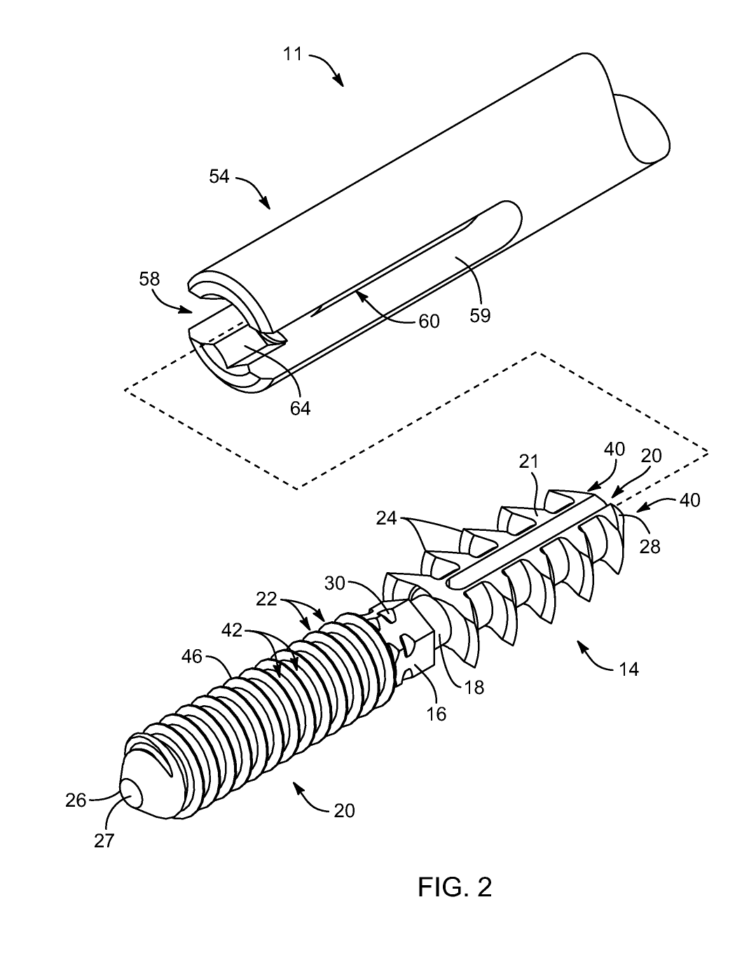

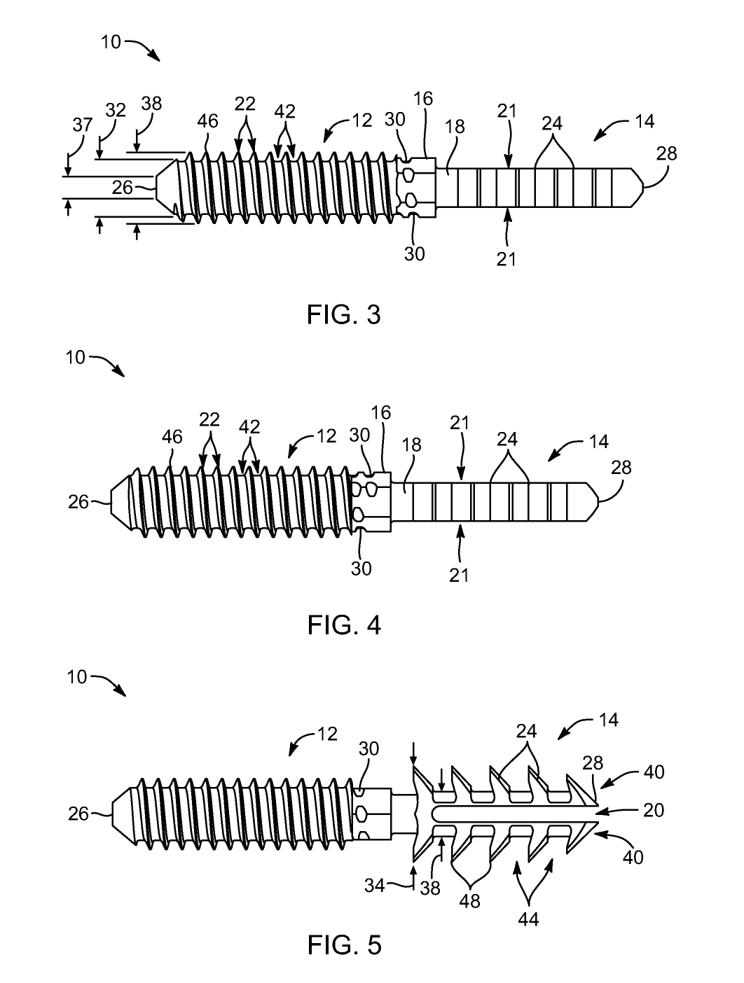

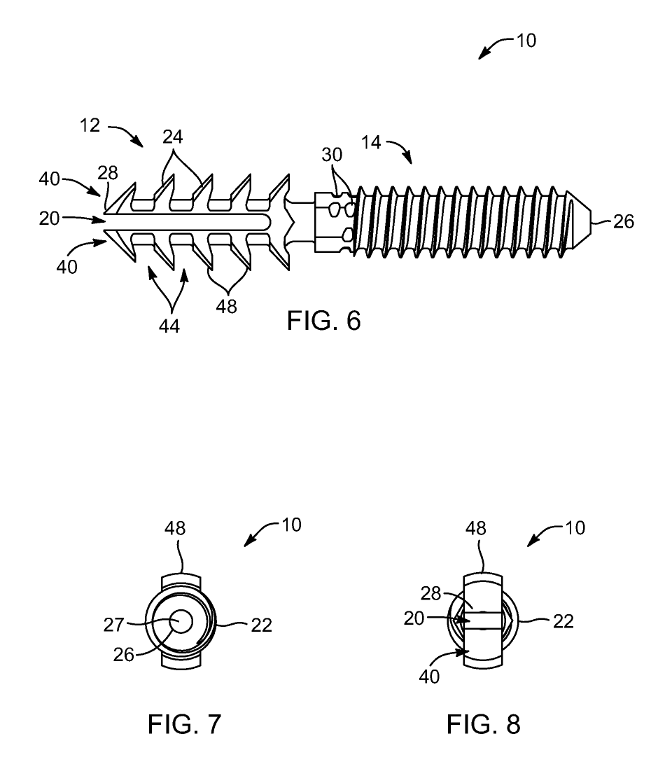

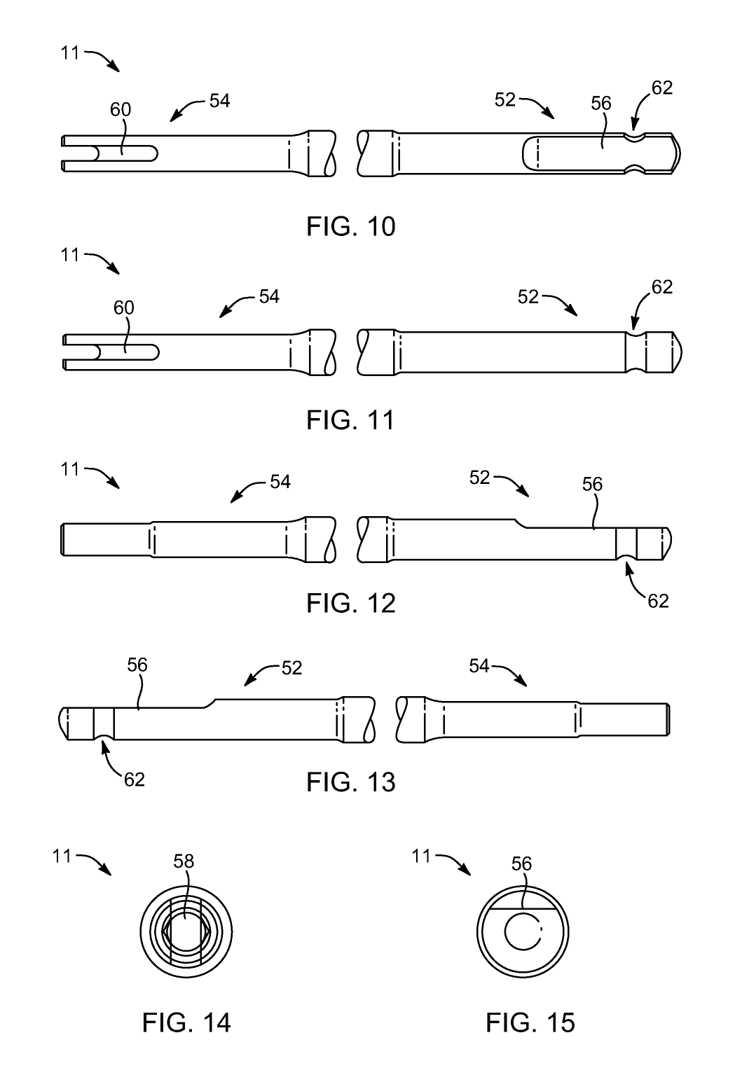

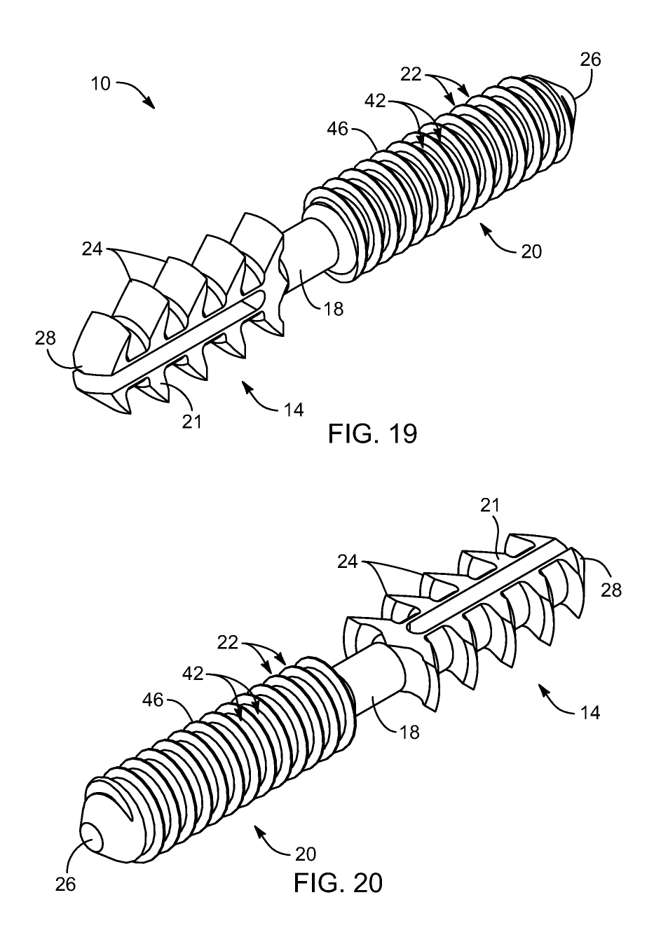

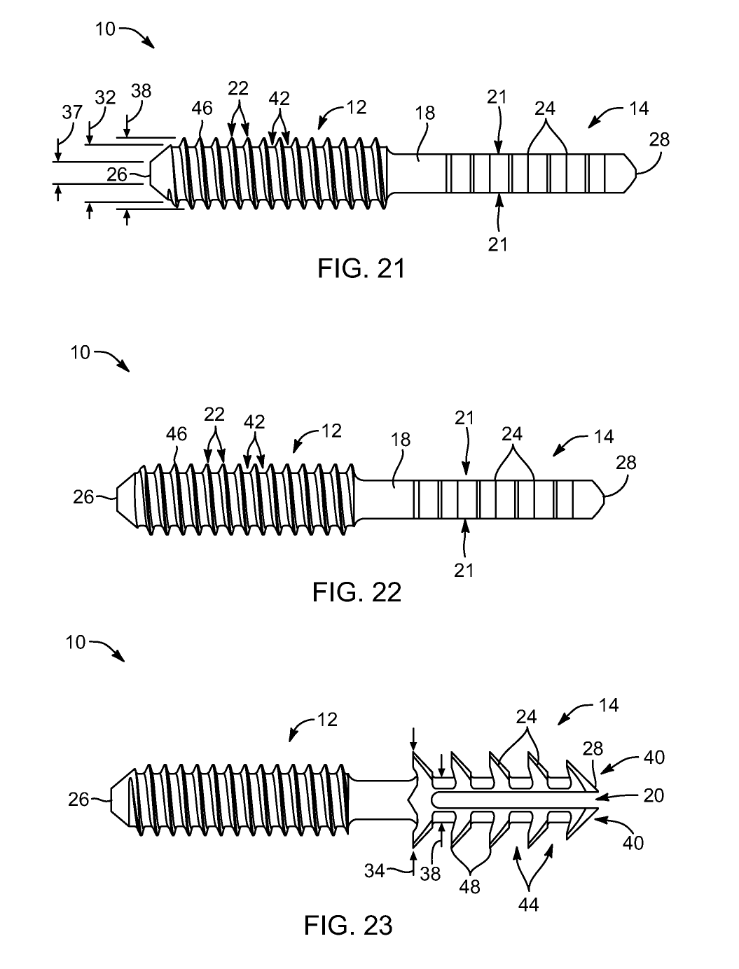

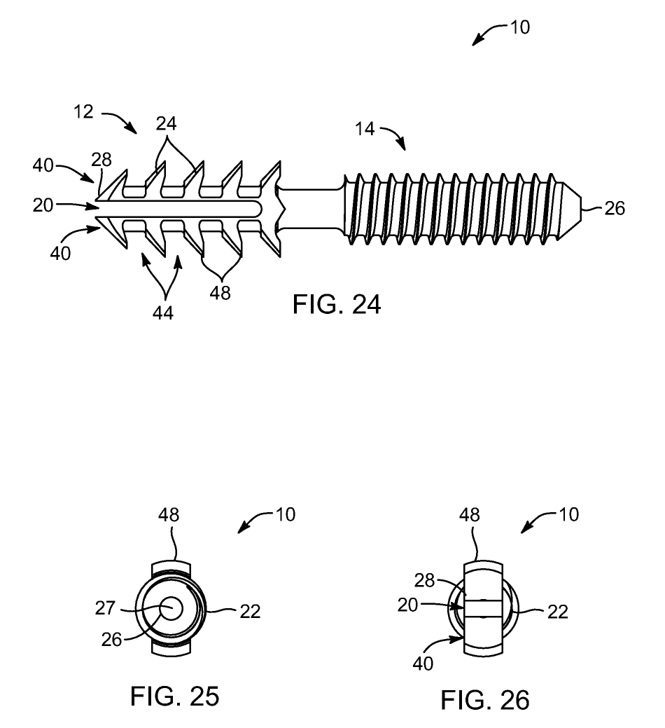

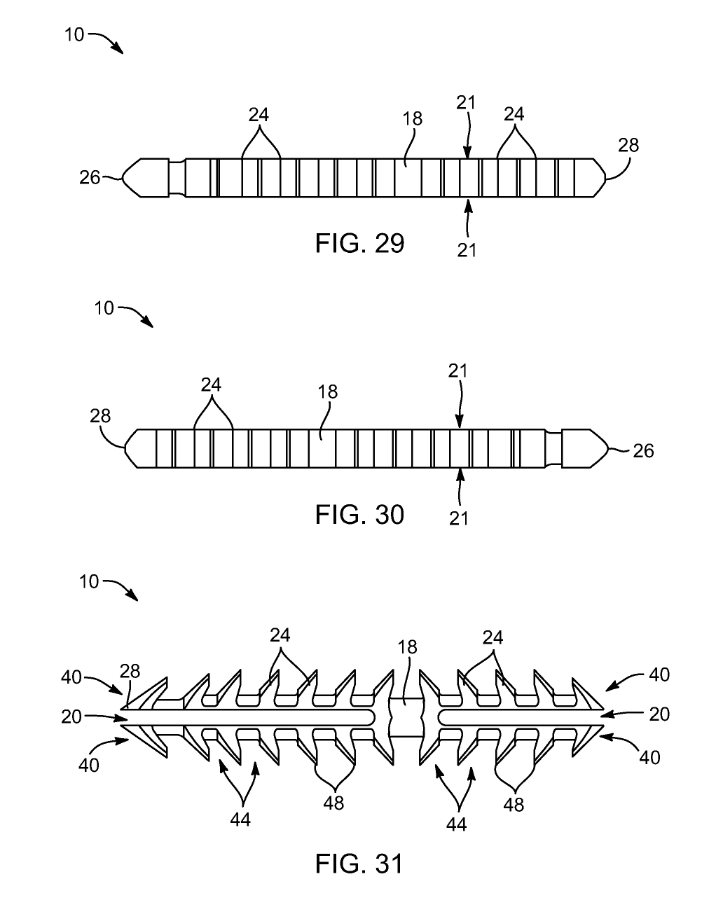

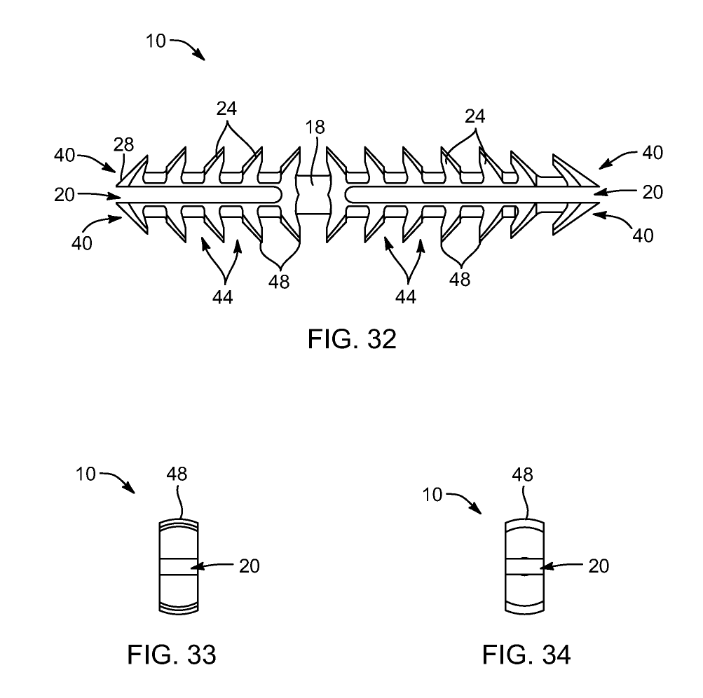

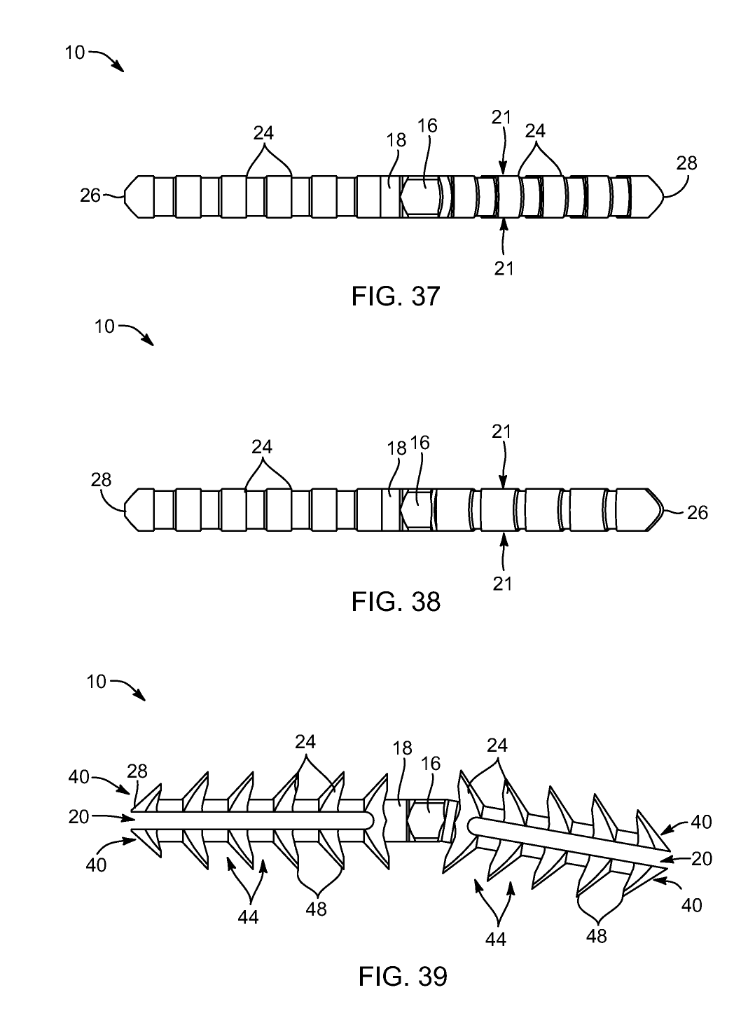

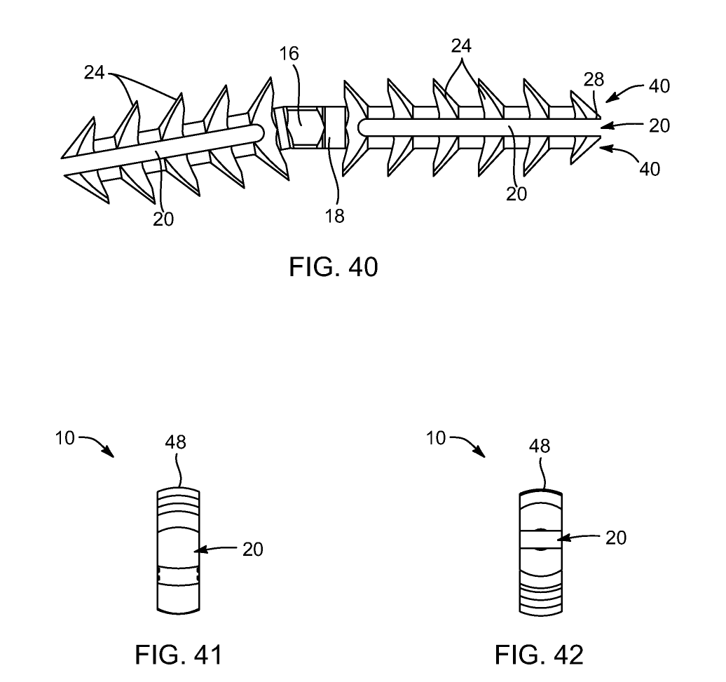

Referring to FIG. 1, while referring generally to FIGS. 1-42, a system 10 or apparatus 10 in accordance with the invention may include an anchor 10 that may be manipulated and applied using a tool 11. The anchor 10 includes a screw portion 12 or a screw 12 formed in a unit with a barb portion 14 or barb 14. In the illustrated embodiment, the screw 12 and barb 14 portions are formed together of a single, homogeneous material simultaneously formed in any suitable manner.

For example, a screw machine may form such devices, but stamping may also produce similar products. Thus, a suitable manufacturing method may apply.

In certain embodiments, the apparatus 10 may be formed to be of a biodegradable material. Nevertheless, in other embodiments, durable materials that are not rejected by the body may be used. For example, stainless steel, titanium, and the like have been found suitable for applications useful for anchors 10 in accordance with the invention.

The anchor 10 may also be formed to include a shank 16 or a shank portion 16. In certain embodiments, it has been found that the shank 16 is best located immediately adjacent the screw portion 12. In application, the screw 12 may be threaded into a portion of bone, typically the medullar portion. Accordingly, the shank 16 may follow the screw 12 into the medullar portion of a bone joint segment with the shank 16 sunk into the bone until the end of the shank 16 farthest from the screw 12 is flush with the surface thereof.

In some embodiments, the shank 16 may be shaped in any suitable shape to stabilize the screw 12 against turning. That is, as healing begins a non-circular shape resists any tendency of rotation between adjacent joints that will be anchored together by the anchor 10. In certain embodiments, the shank 16 may be formed in a hexagonal polygon cross section in order to form edges that are not round. This way, as the tissue grows back to replace itself about the shank 16, the shank 16 is further stabilized, and acts to stabilize the anchor 10.

Likewise, in some embodiments, the shank 16, if formed as a polygon, particularly as a hexagon, may also be used to engage a tool 11. Thus, the shank 16 may operate like a nut or the head of a bolt to engage a tool 11 used to rotate the shank 16, in order to thread the screw 12 into a segment of bone.

In addition to the shank 16, the anchor 10 may include a shaft 18 between the screw portion 12 and the barb portion 14. In the illustrated embodiment, the shaft 18 fits between the shank 16 and the barb portion 14. The length and diameter of the shaft 18 are selected in certain embodiments to support bending. For example, a surgeon in using an anchor 10 in accordance with the invention may determine in advance to bend the shaft 18 after the anchor 10 has been installed, both with the screw portion 12 and the barb portion 14. Accordingly, last in its alignment, whether angular in nature, or whether a closer fit between the contacting adjacent surfaces of bone may be desired, the shaft 18 may be bent in order to provide any last minute adjustments according to the desires and recommendations of an attending surgeon.

In other embodiments, the shaft 18 may be pre-angled in order to provide a known and desired angular difference between the orientation of the screw portion 12 and the barb portion 14 of the anchor 10 once installed. In this way, a pre-determined angle may exist in the shaft 18. If the pre-determined angle is to be further modified, the shaft 18 may still be further bent in any suitable direction. Thus, whether laterally or transversely (at a right angle thereto) with respect to a longitudinal axis of a bone joint, the shaft 18 may be realigned or angled according to the desires of a surgeon.

In the illustrated embodiment, an anchor 10 may include a slot 20. The effect of the slot 20 is to form the barb portion 14 into a cantilevered, pronged arrangement. Also, the slot 20 may serve to engage the tool 11 in order to drive the screw portion 12 into a bone joint for a surgery. The slot 20 may also provide a region in which tissue will reform and fill up, further stabilizing the anchor 10 once in place. Typically, an anchor 10 is not removed. Rather, the anchor 10 stabilizes a surgery, and remains in place after healing is complete.

The screw portion 12 may include threads 22 at a selected pitch, depth, advance angle, and the like. Moreover, the threads 22 may be buttressed such that they tend to be supported more against force or deflection in one direction than another. For example, in one embodiment, the threads 22 may be thought of as flutes that have a longer rise and cross section on the forward end, as the screw 12 advances, and a more precipitous cut, even an undercut, on the side of the thread 22 away from the direction of advancement.

By the same token, the barb portion 14 may include a plurality of barbs 24, the barbs may act on cantilevered arms in order to pass through a pilot hole, and then work out into the medullar region of the bone, ultimately engaging the cortical portion thereof. Likewise, the threads 22 are best engaged into cortical material of the bone, the outer shell like portion that is more dense, stronger, and much harder than the medullar or central region of the marrow.

In order to facilitate an insertion, the screw end 12 or screw 12 may have a point 26. The point 26 may actually not come to a literal physical point, but may be truncated. One reason why a point may not be required is that a pilot hole will typically serve well to minimize trauma, aid healing, and reduce pain. Stress in a bone translates to pain during recovery. Thus, a pilot hole may be drilled in a bone segment, and the point 26 need only accommodate that pilot hole. Accordingly, the point may be sized to engage the pilot hole, which also may be countersunk in order to receive the point 26, whereupon the threads 22 will engage the bone and advance the screw portion 12 thereinto. Also, the screw 12 may be hollow. Thus it would not form a sharp point 26.

Likewise, the barb portion 14 may include a point 28 associated with the first of the barbs 24. Like the opposite end 12 of the anchor 10, the point 28 at the barb portion 14 of the anchor 10 may be sized to fit or otherwise engage a countersink in a pilot hole. Likewise, a pilot hole could be simply drilled at a single diameter, and the barb point 28 may engage that pilot hole.

Referring to FIGS. 1-8, and more generally to FIGS. 1-42, an anchor 10 in accordance with the invention may be provided with cavities 30 configured to receive tissue growth therethrough. Thus, several functions may be met. In one manner, the conservation of mass principle requires that mass be neither created nor destroyed. Typically, except in circumstances of gases, the material will remain comparatively incompressible.

In the illustrated embodiment, the central portion of the screw portion 12 of the anchor 10 may be hollow. Accordingly, the effect of the point 26 of the screw portion 12 is effectively to cut and separate a core of the medullar portion of the bone, which then passes into the inner diameter of the screw portion 12. Therefore, some type of venting is appropriate. The cavities 30 extend from the outer portion of the shank 16 to the inner portion of the screw portion 12 of the anchor 10. Thus, air and liquids may be pistoned or swept through and out of the center portion of the screw 12, exiting by way of the cavities 30 formed in the shank 16.

The screw 12 may have an outer diameter 32 defining the outermost edge of the flutes or threads 22 thereof. Meanwhile, the outermost diameter 34 of the barbed portion 14 will typically follow an arcuate path, although not usually for a full circle. That is, the barbs 24 are formed to present a flat aspect 21 that assists in stabilizing the barbs in place, reduces the requirement in the size of the pilot hole required, and also provides for cantilevering of the barbs 24 in order that they may ultimately extend to their maximum outside diameter 34 to engage the cortical portion of the bone.

Meanwhile, the threads 22 have an inner diameter 36 that defines the valley, trough, or the relief that exists at a lesser diameter than the outer diameter 32 of each of the flutes 22 or threads 22. Moreover, the screw 12 may also have an innermost diameter 37 that represents a cavity formed or a tubular vacancy in the center of the screw 12, so that the screw 12 effectively passes or even cores a portion of the material from the medullar region of the bone upon insertion thereinto.

As with the screw portion 12, the barbed portion 14 has an outer diameter 24 as well as an inner diameter 38. The inner diameter 38 provides for additional material, provides for a smooth and arcuate surface, and provides additional stiffness for the barb portion 14. Effectively, the barb portion 14 is divided into two prongs 40. Each of the prongs 40 contains an array of barbs 24, the first one representing the point 28.

Thereafter, the barbs may increase in diameter or rather have a larger outer diameter 34 than the first barb. Thus, just as each of the threads 22 has an outermost diameter 32 and an inner thread diameter 36, each of the barbs 24 has an outer diameter 34 at which it engages the bone, and an inner diameter 38 that represents effectively the beam that cantilevers or carries the barb 24. That beam is one of the prongs 40 arrayed with barbs 24 there along.

Thus, each of the threads has a gap 42 or pitch 42 between threads. Similarly, each of the barbs 24 has a pitch 44 or gap 44 in which it receives material. That is, the threads 22 actually wend their way through bone material and leave it largely in place and fill in the gaps 42 between the threads 22.

In contrast, the gaps 44 may be less filled with bone material inasmuch as the barbs 24 must pass therethrough. However, the cantilevered effect of the prongs 40 permits the barbs 24 to move toward one another, forced by the pressure of the surrounding bone. Nevertheless, upon the slightest provocation to retreat, or upon coming to rest, the prongs 40 are urged apart by their inherent elasticity, causing each of the barbs 24 to move out toward its edge 48. The edges 46 of the screws 12 or the edges 46 of the threads 22 will cut into and anchor against the bone material, and particularly against cortical material. Likewise, the edges 48 of the barbs 24 will tend to advance outward as they come to rest, cutting through the medullar material and engaging the cortical material of the bone.

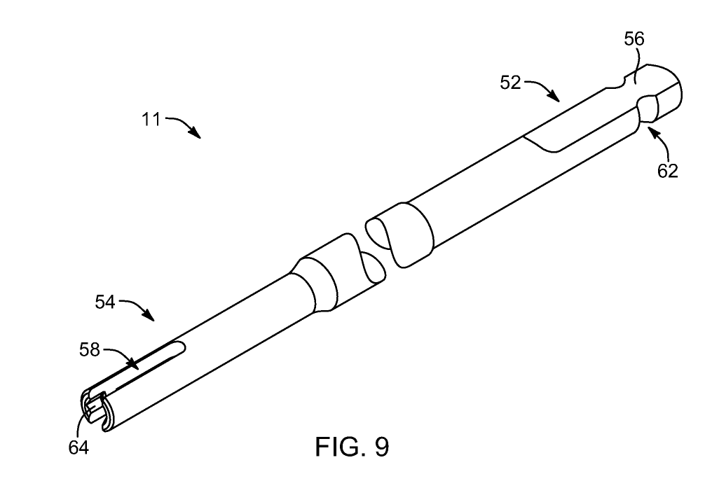

Referring to FIGS. 9-15, while continuing to refer generally to FIGS. 1-42, a tool 11 in accordance with the invention may be formed to fit over the barb portion 14 of an anchor 10. Accordingly, opposite the received portion of the tool 11, a handle portion 52 may be adapted to be gripped by a hand of a surgeon. For example, the handle portion 52 may include a flat 56 as well as relief 62 for gripping.

Any time relief is provided, such as by notching, knurling, or the like, the handle portion 52 may be more readily gripped because flesh from the hand of a user expresses into the slot or relief area providing better than a frictional grip thereon. Accordingly, the tool 11 may include a flat 56, a relief slot 62, knurling, or the like in order to provide better grip of the tool 11 for a surgeon. Accordingly, a surgeon user may thereby rotate with greater security the tool 11 in order to thread the anchor into a bone joint where the anchor 10 will serve to stabilize the conjoining of two joints during healing.

Likewise, the anchor portion 54 or the receiving portion 54 of the tool 11 may include a slot 58 to receive the barb portion 14 of the anchor 10. Specifically, the prongs 40 that form the main beams 40 of the barb portion 14 may fit within the slot 58, sized to receive it.

Similarly, a web 60 may extend between opposite faces of the slot 58 in order to engage the slot 20 in the anchor 10. Thus, the barb portion 14 is engaged on the flats 21 by the opposing faces that form the slot 58. Meanwhile, the slot 20, and the inner faces of the barb portion 14 that form the slot 20, are engaged by the web 60.

Moreover, in certain embodiments, a relief 64 may be formed to receive the hexagonal or other shape of the shank 16. Thus, the anchor 10 may be engaged by the tool 11 by the slot 58 engaging the flats 21 of the barb portion 14, the web 60 engaging the faces of the slot 20 in the barb portion 14, and the relief section 64 or a hexagonal relief 64 fitted to the shank 16 engaging the shank.

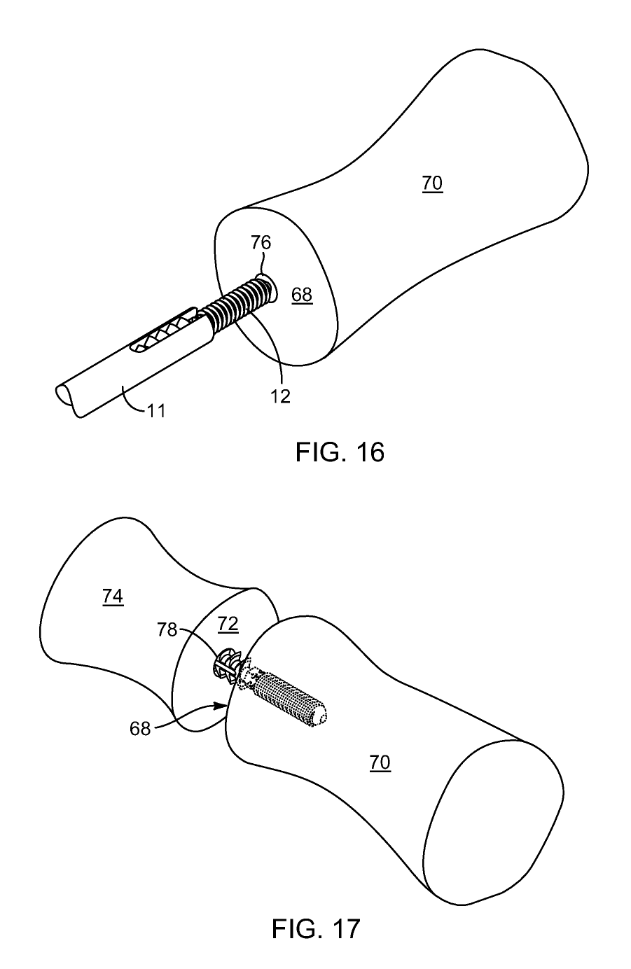

Referring to FIGS. 16-17, while continuing to refer generally to FIGS. 1-42, the use of an anchor 10 and the tool 11 in a surgical operation may include several distinct steps. Some may be done simultaneously, and some may be executed sequentially.

As a preparatory matter, the face 68 of a first bone joint 70 may be trimmed, or resected in order to render the joint 70 capable of joining with another portion of bone. That is, so long as the outermost layer of the joint 70 remains intact, it will not tend to heal with another bone. However, by resecting the face 68 slightly toward the bone joint 70, the face 69 will then be in a condition to heal against another portion of bone. A face 72 of a second bone joint 74 is likewise resected in order to trim it and prepare it to heal against the first joint 70.

Initially, the bone-joints may be drilled with pilot holes. Then, the anchor 10 is placed in the tool 11. The tool is rotated as the screw portion 12, and particularly the point 26 thereof, is urged into the pilot hole of the first bone joint 70. The tool 11 is rotated, by gripping the flat 56 on the handle portion 52.

Accordingly, the threads 22 spiral into the medullar portion of the first bone joint 70, until the shank 16 is effectively buried flush with the surface 68 or face 68 of the joint 70. The tool 11 may then be removed. The tool 11 has engaged the barb portion 14 of the anchor 10, thus using the barb portion 14 as an engagement region or as a head for the engagement by the tool 11 of the screw 12.

Upon withdrawal of the tool 11 along a longitudinal axis of the barb portion 14, the tool 11 may be removed Likewise, as the screw 12 was turning into the face 68 of the joint 70, the face 68 eventually pushes the tool 11 back and off the shank 16, thus disengaging the relief portion 64 of the tool 11 from the shank 16 of the anchor 10.

Once the tool 11 has been withdrawn, the barb portion 14 is exposed and projecting from the joint 70, and particularly from the face 68 thereof. A pilot hole, previously or now drilled into the face 72 of the joint 74 provides a certain amount of relief, and directional piloting of the barb portion 14. Thus, the surgeon may then push the point 28 of the barb portion 14 into the pilot hole.

Thus the screw 12 is initially guided and engaged in the pilot hole 76 in the first joint 70. Meanwhile, at this next stage, the barb portion 14 is driven by force into the pilot 78 in the second joint 74. The cantilevered prongs 40 will move toward one another, thus providing relief on the edges 48 of the barbs 24. However, upon any tendency to come to rest, or to be withdrawn, each of the edges 48 immediately cuts and drives outward due to the undercut shaping thereof. Thus, the barbs 24 provide a positive holding of the second joint 74 against the first joint 70.

At this point, the faces 68, 72 are typically in full plane or contact. Inasmuch as each of the faces 68, 72 is resected, typically by a saw or other tool that is capable of forming a planar surface 68, 72 then alignment of the faces 68, 72 is preferred for best healing. Nevertheless, in order to obtain that alignment, a surgeon may bend the anchor, at the shaft 18 in order to provide that alignment.

Moreover, other considerations may also be at play. The surgeon may determine that slight rotation is necessary, and a rotation of the joint 74, or even of the joint 70 will provide rotation of the screw portion 12 with respect to the joint 74. Thus, rotational alignment may be modified slightly if desired. Moreover, any type of lateral or transverse alignment, that is, alignment movements in any radially direction or orthogonal to the longitudinal axis of the anchor 10 may also be made by bending the shaft 18.

The shaft 18 may be bent by simply providing relative motion in any radial direction between the joint 70 and the joint 74, by the manipulation of either one with respect to the other. Thus, one may be held still while the other is angled. In this manner, an anchor 10 in accordance with the invention may provide additional benefits by providing last minute inspection of the surgery, and slight modifications or the angular relationships and rotational relationships between the joints 70, 74, in order to obtain the best result.

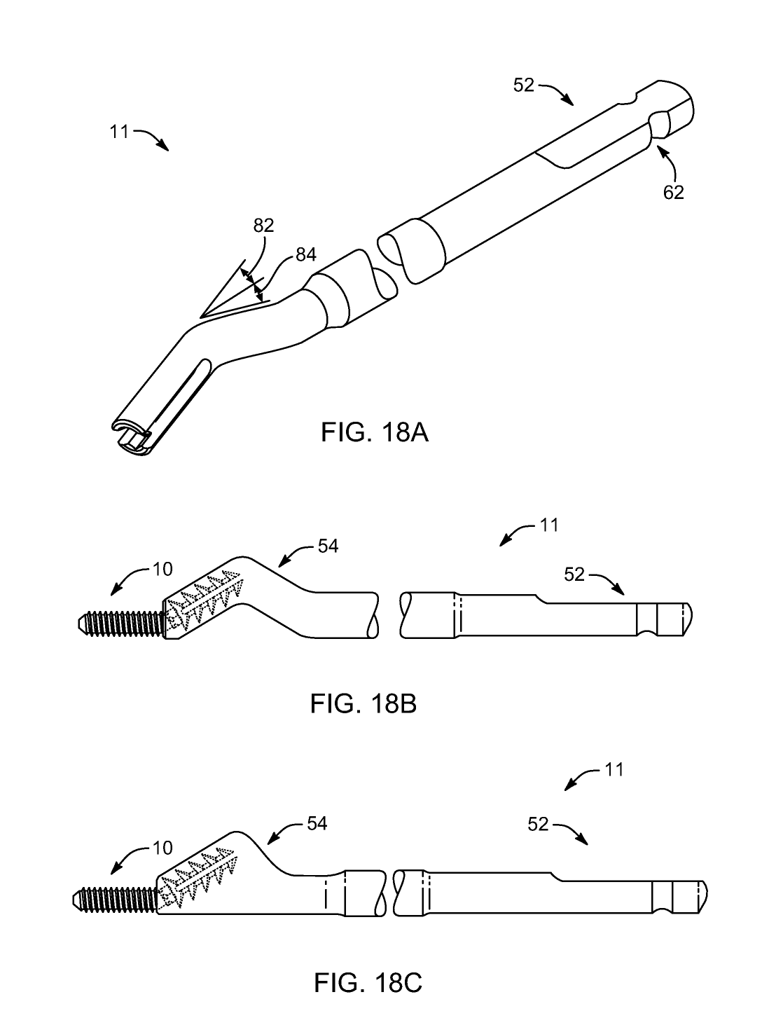

Referring to FIGS. 18A-18C, while continuing to refer generally to FIGS. 1-42, in an apparatus and method in accordance with the invention, a tool 11 may be formed to service an anchor that has a pre-determined offset angle between the longitudinal axis of the screw portion 12, and the longitudinal axis of the barb portion 14 of the anchor 10. In one embodiment of an apparatus and method in accordance with the invention, a tool 11 may be formed to receive an anchor 10 having a pre-determined angle of bending of the shaft 18 thereof.

Thus, two angles 82, 84 may be formed near the anchor portion 54 of the tool 11. A first angle 82 provides the angle required to accommodate a barb portion 14 of an anchor 10. Placing an anchor 10 into the anchor portion 54, and the slot 58 particularly, of the tool 11, the screw portion 14 is aligned with the handles 52. The longitudinal axis thereof is in alignment, and in fact collinear with, the longitudinal axis of the handle portion 52 of the tool 11.

In order to connect the anchor portion 54 of the tool 11 to the handle portion 52, an angle 84 opposite the angle 82 must be made in the tool 11. This takes back out of the tool 11 the angle 82 included for accommodation of the barb portion 14. This is an equal and opposite offset angle from the longitudinal axis of the screw portion 12. Upon rotation of the handle portion 52 by a surgeon, the tool 11 rotates about its own longitudinal axis, which is collinear with the longitudinal axis on the screw portion 12. Upon completing of the threading portion of the process of inserting the anchor 10, a surgeon may draw the handle portion 52 away from the anchor 10, and thus remove the slot 58 and web 60 from engagement with the barb portion 14 of the anchor 10.

Referring to FIGS. 18A-18C, the tool 11 may be conformed to the procedures of placing an anchor having a pre-determined angle offsetting the screw portion 12 from the barb portion 14. Thus, in FIGS. 18A, 18B, a rod may be formed with two bends, at the respective angles 82, 84 in order to position the screw portion 12 of an anchor 12 in alignment with the handle portion 52.

However, line of sight alignment may be enhanced if the handle progresses continuously in the longitudinal direction. Thus, with the screw portion 12 extending therefrom as in FIG. 18C, natural line of sight and natural eye-to-hand coordination may aid orientation of the tool 11. The additional clearance during a rotational sweep of the tool 11 as it threads the screw portion 12 into a joint 70, 72 will effectively be the same in all such embodiments.

Extraction of the tool 11 after installation of the anchor is along the longitudinal axis of the barb portion 14. For example, the slot 58 of the tool disengages from the flats 21 of the barbs 24, while the slot 20 of the barb portion 14 disengages from the web 60 of the tool 11.

Referring to FIGS. 19-26, in some embodiments of an apparatus and method in accordance with the invention, an anchor 10 need not have the shank 16. In this embodiment, the flats 21 on the barbs 24 are engaged by the slot 58 of the tool, and the slot 20 of the anchor 10 is engaged by the web 60 of the tool. However, the shank 16, being absent, is not engaged by the relief 64 in the tool 11. The installation of such an embodiment progresses the same as that of previously described embodiments.

Stability against rotation is provided by the flats 21 of the barbed portion 14. Stability in a longitudinal direction is provided by the threads 22 and the barbs 24. Final adjustments in a longitudinal direction may be made by advancing the joints together, further advancing the barbs 24. Rotational adjustments may be made by rotating the joint holding the barb portion 14 with respect to the joint holding the screw portion 12. Angular adjustments may be made by bending the anchor at the neck 18 or shaft portion 18.

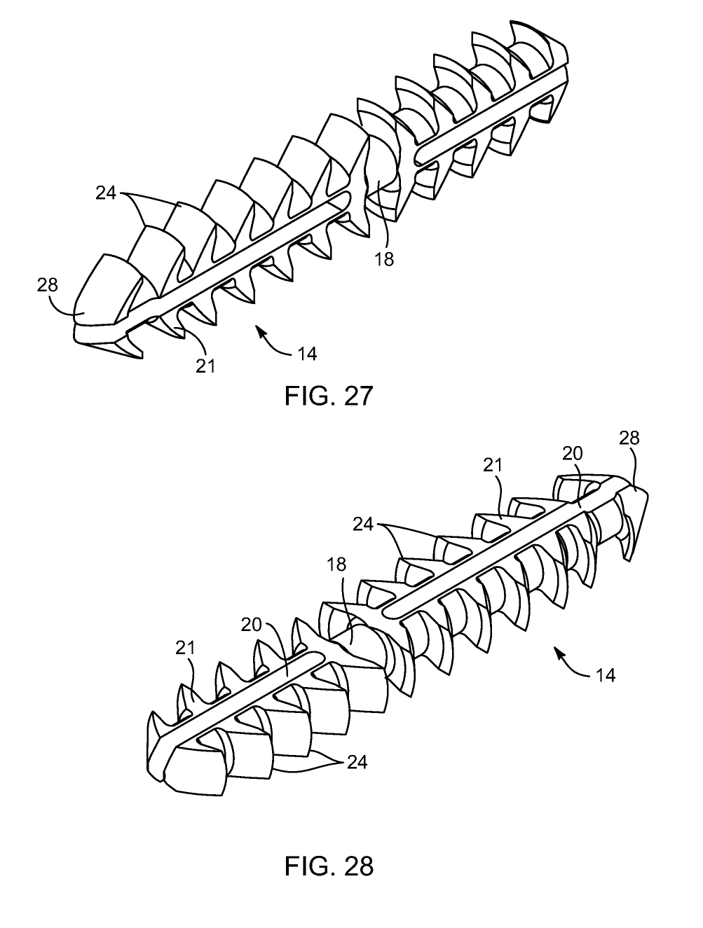

Referring to FIGS. 27-34, in certain embodiments, an anchor may include both ends as barbed portions 14. In this embodiment, insertion of the anchor with the tool 11 may by done by a longitudinal push of the first set of barbs 24 into the pilot hole 76 in response to force applied to the tool 11. Thereafter, the second set of barbs 24 may be inserted into the second pilot hole 78 by pushing the pilot hole 78 in the joint 70 onto the second set of barbs 24. Rotational adjustment is more difficult and damaging, unless accomplished before setting the second set of barbs 24. Angular adjustments may be made by bending at the neck 18.

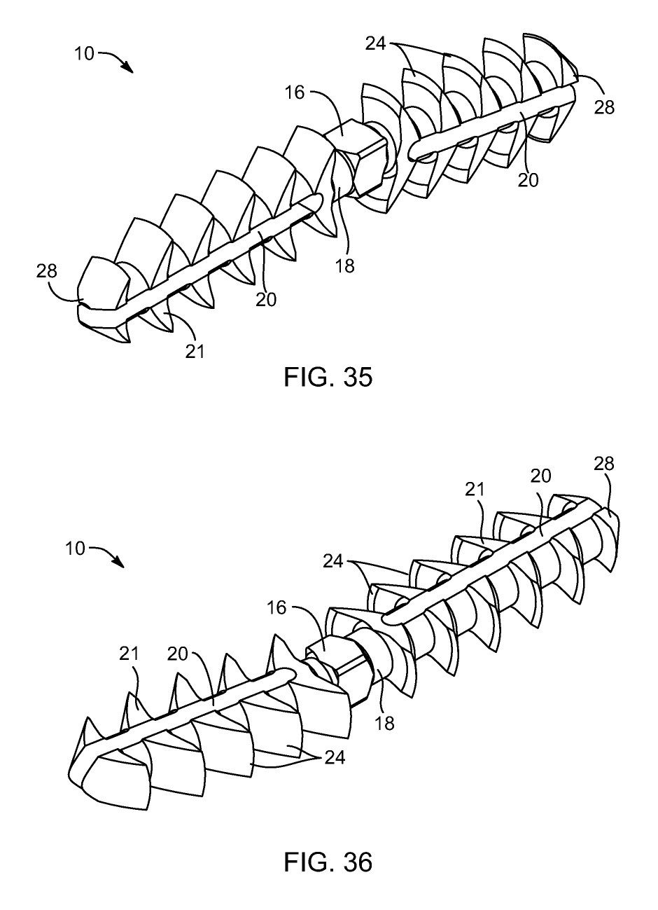

Referring to FIGS. 35-42, a pre-set angle may be formed in the anchor 10, regardless of which types of ends 12, 14 are used. Accordingly, the apparatus and method of this embodiment may be employed in a manner similar to that for the embodiment of FIGS. 17-24. Typical preset angles may be used to accommodate a need for modifying the relative angle between two joints.

The present invention may be embodied in other specific forms without departing from its spirit or essential characteristics. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

What is claimed and desired to be secured by United States Letters Patent is:

1. A method of arthrodesis, the method comprising:
providing an anchor, comprising a monolithic, homogeneously formed structure, defining a longitudinal axis and a radial direction orthogonal thereto, the anchor comprising
a first end comprising threads,
a second end comprising prongs extending longitudinally and provided with barbs extending radially therefrom, and
a shank between the first and second ends shaped to have a variation in radius from the longitudinal axis to the circumference thereof;
accessing the head of a proximal phalangial joint of a subject;
accessing the base of an intermediate phalangial joint of the subject;
forming a first interface surface by resecting the head
forming a second interface surface by resecting the base;
drilling a first pilot in the head through the first interface surface;
drilling a second hole in the base through the second interface surface;
threading the first end into one of the first and second pilots; and
placing the first and second interface surfaces in contact by urging the second end into the other of the first and second pilots.

2. The method of claim 1, wherein:
the prongs are cantilevered between a base region thereof proximate the first end and a separation region proximate the second end; and
the barbs each comprise an edge facing radially outward from the longitudinal axis.

3. The method of claim 1, wherein the proximal and intermediate joints each comprise a cortical portion, formed of a comparatively harder and stronger material proximate an outer boundary thereof, and a medullar portion, formed of a comparatively softer and weaker material proximate a central axis thereof, the method further comprising:
urging the prongs toward the longitudinal axis due to compression of the barbs by pressure from at least the medullar portion acting thereon; and
urging of the edges of the barbs, by cantilevered loads from the prongs, to engage the cortical portion.

4. The method of claim 1, wherein the anchor further comprises a shaft between the first and second ends having a length and diameter sized to admit to bending.

5. The method of claim 4, further comprising:
orienting the first and second interface surfaces with respect to one another by bending the shaft; and
urging the first and second interface surfaces together by applying an axial load to the intermediate phalangial joint along the central axis thereof.

6. The method of claim 5, further comprising:
compressing of the prongs toward the longitudinal axis in response to movement of the barbs with respect to the medullar region; and
urging of the barbs away from the longitudinal axis by residual bending forces remaining in the prongs from displacement in response to the compressing.

7. The method of claim 6, further comprising engaging of the cortical portion by the edges of the barbs in response to the urging of the barbs away from the longitudinal axis.

8. The method of claim 1, wherein:
the prongs are cantilevered between a base region thereof proximate the first end and a separation region proximate the second end;
the barbs each comprise an edge facing radially outward from the longitudinal axis;
the anchor further comprises a shaft between the first and second ends formed in a bend creating an offset angle between the longitudinal axis at the first end and the longitudinal axis at the second end;
the proximal and intermediate joints each comprise a cortical portion, formed of a comparatively harder and stronger material proximate an outer boundary thereof, and a medullar portion, formed of a comparatively softer and weaker material proximate a central axis thereof;
the method further comprises:
urging the prongs toward the longitudinal axis due to compression of the barbs by pressure from at least the medullar portion acting thereon; and
urging of the edges of the barbs, by cantilevered loads from the prongs, to engage the cortical portion.

9. The method of claim 8, wherein the shaft has a length and diameter sized to admit to bending.

10. The method of claim 9, further comprising:
orienting the first and second interface surfaces with respect to one another by bending the shaft; and
urging the first and second interface surfaces together by applying an axial load to the intermediate phalangial joint toward the proximal phalangial joint.

11. The method of claim 10, further comprising:
compressing of the prongs toward the longitudinal axis in response to movement of the barbs with respect to the medullar region; and
urging of the barbs away from the longitudinal axis by residual bending forces remaining in the prongs from displacement in response to the compressing.

12. The method of claim 11, further comprising engaging of the cortical portion by the edges of the barbs in response to the urging of the barbs away from the longitudinal axis.

13. A method comprising:
providing an anchor, comprising
a first portion comprising at least one of threads and first barbs,
a second portion comprising prongs extending longitudinally and provided with second barbs extending radially therefrom,
a neck disposed between and interconnecting the first and second portions;
the first and second portions and neck being formed as a monolithic, homogeneously formed unit, defining at least one longitudinal axis and radial direction orthogonal thereto;
providing a tool having a receiving portion shaped to receive at least one of the first and second portions
accessing the head of a proximal phalangial joint of a subject;
accessing the base of an intermediate phalangial joint of the subject;
placing the anchor in the tool;
applying force to the anchor through the tool;
penetrating the first portion into one of the head and the base by the applying force;
removing the tool to leave the anchor emplaced;
penetrating the second portion into the other of the head and base by urging the base toward the head in a linear translation; and
placing the head and base into mutual contact by the urging.

14. The method of claim 13, further comprising:
forming a first interface surface by resecting the head; and
forming a second interface surface by resecting the base.

15. The method of claim 13, wherein the neck has a circular cross section and radius sized for bending without damage to bone penetrated by the first and second portions, the method further comprising finalizing alignment of the proximal and intermediate phalangial joints by bending the neck.

16. The method of claim 15, further comprising:
drilling a first pilot in the head through the first interface surface; and
drilling a second hole in the base through the second interface surface.

17. The method of claim 16, wherein
the applying force to the anchor through the tool penetrates the first portion into the head; and
the linear translation penetrates the second portion into the base.

18. The method of claim 13, wherein
the prongs are provided with barbs extending radially therefrom
the providing further comprises providing a shank between the first and second portions shaped to engage the tool in rotation about the at least one longitudinal axis.

19. The method of claim 13, wherein the proximal and intermediate phalangial joints each comprise a cortical portion, formed of a comparatively harder and stronger material proximate an outer boundary thereof, and a medullar portion, formed of a comparatively softer and weaker material proximate a central axis thereof, the method further comprising:
deflecting the prongs toward the longitudinal axis due to compression of the second barbs by pressure from at least the medullar portion acting thereon;
moving the edges of the second barbs, by cantilevered loads from the prongs, to engage the cortical portion; and
realigning the first and second portions by bending the neck therebetween.

20. An apparatus formed as an arthrodesis anchor, the apparatus comprising:
a first portion comprising at least one of threads and first barbs;
a second portion comprising second barbs;
the second portion, further comprising at least two prongs extending longitudinally and having the second barbs extending radially therefrom;
a neck disposed between the first and second portions and having a diameter sized to bend under lateral force applied by first bone material enveloping the first portion and second bone material enveloping the second portion without perceptible damage to the first and second bone materials; and
the first and second portions and the neck being a monolithic, homogeneously formed unit, defining a longitudinal axis, and a radial direction orthogonal thereto.
